(19) United States
(12) Patent Application Publication
(10) Pub. No.: US 2008/0234486 A1
(43) Pub. Date: Sep. 25, 2008

(54) Novel Processes for the Production of Amorphous Rabeprazole Sodium

(76) Inventors: Elisabeth Schuler Civit, Barcelona (ES); Ana Gavalda Escude, Barcelona (ES)

Correspondence Address: FROMMER LAWRENCE & HAUG, 745 FIFTH AVENUE- 10TH FL., NEW YORK, NY, 10151, US

(21) Appl. No.: 11/997,587

(22) PCT Filed: Aug. 1, 2006
(86) PCT No.: PCT/IB2006/003385

§ 371 Date: Feb. 1, 2008

Related U.S. Application Data
(60) Provisional application No. 60/704,941, filed on Aug. 2, 2005.

(51) Int. Cl. C07D 401/12

(52) U.S. Cl. 546/273.7

(57) ABSTRACT

The invention is directed to overcome the problems associated with the formation of rabeprazole sodium, i.e. formation of (2-({[4-(3-methoxypropoxy)-3-methylpyridin-2-yl]methyl}sulfonyl)-1H-benzimidazole side product which is achieved by a process for the preparation of amorphous rabeprazole salt, e.g. sodium, comprising the steps of:
i) contacting rabeprazole salt complex, e.g. sodium acetone complex with a first solvent system;
ii) filtering the solid from the solvent system used in step i) or distilling, totally or partially, the solvent system used in step i) under reduced or atmospheric pressure, to thereby obtain a residue;
iii) contacting the solid or the residue of step ii) with a second solvent system;
v) filtering the solid from the solvent system used in step iii) or distilling, totally or partially, the solvent system used in step iii) under reduced or atmospheric pressure to thereby obtain a solid;
v) optionally repeating steps iii) and iv) one or more times;
vi) optionally filtering to obtain a wet solid; and
vii) drying the wet solid.

### INCORPORATION BY REFERENCE

Any foregoing applications, and all documents cited therein or during their prosecution (“application cited documents”) and all documents cited or referenced in the application cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

The present invention relates to novel and efficient processes for the production of amorphous rabeprazole sodium.

### BACKGROUND OF THE INVENTION

Rabeprazole sodium is the common name for 2-({[4-(3-Methoxypropoxy)-3-methylpyridin-2-yl]methyl}sulfinyl)-1H-benzimidazole sodium salt, of formula (I).

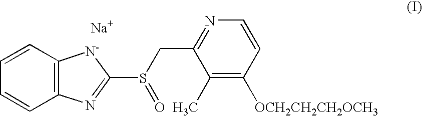

Rabeprazole sodium is an inhibitor of the gastric proton pump. It belongs to a class of antisecretory compounds that do not exhibit anticholinergic or histamine H2-receptor antagonist properties, but suppress gastric acid secretion by inhibiting the gastric H+, K+ ATPase at the secretory surface of the gastric parital cell. Rabeprazole blocks the final step of gastric acid secretion.

Example 33 of U.S. Pat. No. 5,045,552 involves a process for obtaining rabeprazole sodium (see scheme 1) by oxidation of 2-({[4-(3-methoxypropoxy)-3-methylpyridin-2-yl]methyl}thio)-1H-benzimidazole (Compound II) with m-chloroperbenzoic acid in dichloromethane at −45° C. After the completion of the reaction triethylamine was added and the obtained mixture was heated to −10° C., followed by the addition of saturated aqueous solution or sodium carbonate. The mixture was extracted with dichloromethane, dried over magnesium sulfate and the solvent was removed by evaporation to obtain 2-({[4-(3-Methoxypropoxy)-3-methylpyridin-2-yl]methyl}sulfinyl)-1H-benzimidazole (compound III, rabeprazole acid) (see Scheme I). To this crude product aqueous sodium hydroxide solution is added to obtain a solution. This solution was distilled together with ethanol twice and dried in vacuum. Finally, ether was added to the residue to precipitate rabeprazole sodium as a white crystal. The melting point of the disclosed rabeprazole sodium salt is 140-141° C. (d).

Japanese patent application JP2001039975 indicates that the product obtained by example 33 of U.S. Pat. No. 5,045,552 with a melting point of 140-141° C. (d) corresponds to amorphous_rabeprazole sodium. In this application, the X-ray powder diffraction pattern of the amorphous rabeprazole sodium is shown.

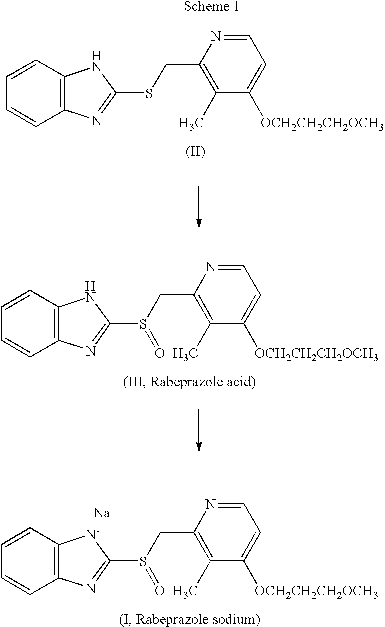

Reference example 2 of U.S. Patent Application Publication US2004/0180935A1 refers to a process for production amorphous rabeprazole sodium by dissolving rabeprazole acid (compound III) in a mixture of sodium hydroxide and methanol at 25-35° C. The solvent is removed by evaporation and the product is precipitated by adding petroleum ether. The precipitated solid is filtered, washed with petroleum ether and dried at 50-60° C. for 12 hours.

A problem of the process referred in Example 33 of U.S. Pat. No. 5,045,552 (see scheme 1) is that during the oxidation of the thioether (compound II), the obtained sulfoxide (compound III) suffers a further oxidation generating the undesired sulfone—(2-({[4-(3-methoxypropoxy)-3-methylpyridin-2-yl]methyl}sulfonyl)-1H-benzimidazole, compound IV,

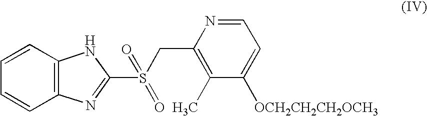

Because of the similar physicochemical properties between the rabeprazole and the sulfone impurity IV, the purification of the final product is very difficult.

U.S. Pat. No. 6,180,652 concerns processes for purification of the rabeprazole sodium from its sulfone impurity performing an acetone complex (compound V). The acetone complex is characterized by X-ray powder diffraction pattern, IR and ¹H-NMR.

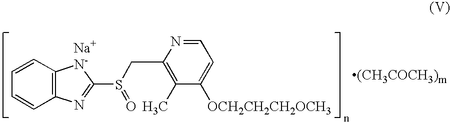

However, it is known that an acetone complex is not suitable for the manufacture of a medicinal compound. So there exists a need in the art to convert the purified rabeprazole sodium acetone complex into a rabeprazole sodium suitable for the manufacture of a medicinal compound.

In the Reference Example 1 of U.S. Pat. No. 6,180,652 amorphous rabeprazole sodium is obtained by lyophilizing (freeze-drying) an aqueous solution of rabeprazole sodium acetone complex.

However, lyophilization is a technique which is not suitable for production at industrial scale because this process presents serious limitations on cost, time, equipment capability and environmental protection.

WO2004/085424A1 refers to the conversion of the rabeprazole sodium acetone complex into amorphous rabeprazole sodium by heating at elevated temperature, preferably between 100 and 110° C. It is well known that exposing rabeprazole-type compounds to high temperatures increases the risk of decomposition to form impurities and as such, heat treatment of rabeprazole sodium acetone complex into amorphous rabeprazole sodium is not adequate for the production of a rabeprazole which is suitable for pharmaceutical use.

Accordingly there are problems in the art involving methods for preparing rabeprazole salts, especially sodium salts.

### SUMMARY OF THE INVENTION

An object of the invention is to address problems associated with the preparation of rabeprazole sodium, i.e. problems with the preparation of (2-({[4-(3-methoxypropoxy)-3-methylpyridin-2-yl]methyl}sulfonyl)-1H-benzimidazole side product.

This object of the invention is achieved by a process for the preparation of amorphous rabeprazole sodium comprising the steps of:

i) contacting rabeprazole sodium acetone complex with a first solvent system;

ii) filtering to obtain a wet solid from the solvent system used in step i) or distilling, totally or partially, the solvent system used in step i) under reduced or atmospheric pressure, to thereby obtain a residue;

iii) contacting the wet solid or the residue of step ii) with a second solvent system;

iv) filtering to obtain a wet solid from the solvent system used in step iii) or distilling, totally or partially, the solvent system used in step iii) under reduced or atmospheric pressure to thereby obtain a residue;

v) optionally repeating steps iii) and iv) one or more times;

vi) optionally filtering to obtain a wet solid; and

vii) drying the wet solid.

An embodiment of the first solvent system includes but is not limited to a hydrocarbon solvent or an ether solvent or an alcohol solvent or mixtures thereof. An embodiment of the second solvent system includes but is not limited to a hydrocarbon solvent or an ether solvent.

Surprisingly, the process of preparing amorphous rabeprazole sodium allows for higher levels of purity with fewer process steps (which minimizes loss of yield of the amorphous rabeprazole sodium). In addition, this purity and/or yield level is achieved without exposing the amorphous rabeprazole sodium to rigorous temperature conditions (e.g. drying at temperatures between 100 and 110° C.).

For the purposes of this invention, rabeprazole salts refers to a salt formed from any positively charged cation (e.g. lithium, sodium, potassium, ammonium, etc.) in association with the negatively charged nitrogen of the benzimidazole ring of the rabeprazole compound. In addition, solvents described in this invention are normally liquid at standard temperature and pressure (room temperature (20-25° C.) and 1 atm of pressure).

Citation or identification of any document in this application is not an admission that such document is available as prior art to the present invention.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are apparent from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1—shows the x-ray diffractogram pattern of rabeprazole sodium acetone complex;

FIG. 2—shows the IR spectrum of rabeprazole sodium acetone complex;

FIG. 3—shows the x-ray diffractogram pattern of amorphous rabeprazole sodium obtained according to the present invention;

FIG. 4—shows the IR spectrum of amorphous rabeprazole sodium obtained according to the present invention;

### DETAILED DESCRIPTION

The present invention is directed to a process for the production of amorphous rabeprazole salt, especially sodium salt, by simple and efficient treatments of the RBZ acetone complex with one or more organic solvents.

A first embodiment of the invention encompasses a process for the preparation of amorphous rabeprazole salt, e.g. sodium, comprising the steps of:
i) contacting rabeprazole salt complex, e.g. sodium acetone complex with a first solvent system;
ii) filtering the solid from the solvent system used in step i) or distilling, totally or partially, the solvent system used in step i) under reduced or atmospheric pressure, to thereby obtain a residue;
iii) contacting the solid or residue of step ii) with a second solvent system;
iv) filtering to obtain a wet solid from the solvent system used in step iii) or distilling, totally or partially, the solvent system used in step iii) under reduced or atmospheric pressure to thereby obtain a wet solid;
v) optionally repeating steps iii) and iv) one or more times; and
vi) drying the wet solid.

The first and second solvent systems can be as in other embodiments herein disclosed. In this embodiment, the first solvent system includes but is not limited to a hydrocarbon solvent or an ether solvent or an alcohol solvent or mixtures thereof. A suitable hydrocarbon solvent includes but is not limited to n-pentane, n-hexane and n-heptane and isomers or mixtures thereof, cyclohexane, toluene and xylene. In an advantageous embodiment, the hydrocarbon solvent is n-heptane. A suitable ether solvent includes but is not limited to diethylether, tert-butyl methyl ether and cyclic ethers such as tetrahydrofuran and 1,4-dioxane. In an advantageous embodiment, the ether solvent is tert-butyl methyl ether. A suitable alcohol solvent includes but is not limited to C₁ to C₄ straight or branched chain alcohol solvents or mixtures thereof (such as methanol, ethanol, propanol and butanol), preferably, methanol, 2-propanol or mixtures thereof.

An embodiment of the second solvent system includes but is not limited to a hydrocarbon solvent or an ether solvent. A suitable hydrocarbon solvent includes but is not limited to n-hexane and n-heptane and isomers or mixtures thereof, cyclohexane, toluene and xylene. In an advantageous embodiment, the hydrocarbon solvent n-heptane. A suitable ether solvent includes but is not limited to tert-butyl methyl ether and cyclic ethers such as tetrahydrofuran and 1,4-dioxane. Advantageously, the ether solvent is tert-butyl methyl ether.

In another embodiment of the solvent systems, the first and second solvent systems may be the same or different. In a preferred embodiment of the solvent systems, the first solvent is an alcohol solvent or mixture of alcohols, preferably methanol and 2-propanol and the second solvent system is a hydrocarbon solvent, preferably n-heptane.

A second embodiment of the present invention provides a process for the preparation of amorphous rabeprazole salt, e.g. sodium, comprising the steps of:
i) suspending rabeprazole salt complex, e.g. sodium acetone complex with a first solvent system;
ii) filtering the solvent system used in step i) or distilling, totally or partially, the solvent system used in step i) under reduced or atmospheric pressure to obtain a wet solid;
iii) optionally repeating steps i) and ii) one or more times; and
iv) drying the wet solid.

The first solvent system can be a solvent system as in any other embodiment of the invention herein disclosed and includes but is not limited to a hydrocarbon solvent or an ether solvent or mixtures thereof. A suitable hydrocarbon solvent includes but is not limited to n-hexane and n-heptane and isomers or mixtures thereof, cyclohexane, toluene and xylene. Advantageously, the hydrocarbon solvent is n-heptane. A suitable ether solvent includes but is not limited to tert-butyl methyl ether and cyclic ethers such as tetrahydrofuran and 1,4-dioxane. Advantageously, the ether solvent is tert-butyl methyl ether.

In a preferred embodiment for step iii), steps i) and ii) are repeated once.

In a third embodiment of the present invention there is provided a process for the preparation of amorphous rabeprazole salt, e.g. sodium, comprising the steps of:
i) dissolving rabeprazole salt complex, e.g. sodium acetone complex with a first solvent system;
ii) optionally decolorizing and/or filtering the solution;
iii) distilling, totally or partially, the solvent system used in step i) under reduced or atmospheric pressure to obtain a residue;
iv) contacting the residue with a second solvent system;
v) distilling, totally or partially, the solvent system used in step iv) under reduced or atmospheric pressure;
vi) optionally repeating steps iv) and v) one or more times;
vii) filtering to obtain a wet solid; and
viii) drying the wet solid.

In this third embodiment, the first and second solvent systems can be as in any other embodiment herein disclosed, e.g., as in the first or second solvent systems. An embodiment of the first solvent system includes but is not limited to one or more alcohol solvent. The alcohol solvent can be, but is not limited to a C₁ to C₄ straight or branched chain alcohol solvent. Advantageously, the alcohol solvent system is methanol or 2-propanol or mixtures thereof. An embodiment of the second solvent system includes but is not limited to a hydrocarbon solvent or an ether solvent. The hydrocarbon solvent can be but is not limited to n-hexane and n-heptane and isomers or mixtures thereof, cyclohexane, toluene and xylene. The hydrocarbon solvent advantageously is n-heptane. The ether solvent can be but is not limited to a tert-butyl methyl ether and cyclic ethers such as tetrahydrofuran and 1,4-dioxane. The ether solvent advantageously is tert-butyl methyl ether. Thus, advantageous embodiments of the second solvent system are n-heptane or tert-butyl methyl ether or mixtures thereof.

In preferred embodiment of the third embodiment of the present invention is a process for the preparation of amorphous rabeprazole salt, e.g. sodium, comprising the steps of:
i) dissolving rabeprazole salt complex, e.g. sodium acetone complex, with a first solvent system comprising methanol and isopropanol to form a solution;
ii) decolorizing and filtering the solution;
iii) distilling totally, the solvent system used in step i) under reduced or atmospheric pressure to obtain a residue;
iv) contacting the residue with a second solvent system comprising heptane;
v) distilling, totally or partially, the solvent system used in step iv) under reduced or atmospheric pressure;
vi) repeating steps iv) and v) one or more times;
vii) filtering, thereby obtaining a wet solid; and
viii) drying the wet solid.

The solution of the rabeprazole salt complex, e.g. sodium acetone complex, obtained in step i) can be treated with a decolorizing agent, if desired, in order to improve quality attributes such as color especially if there is a low amount or an absence of impurities (e.g., an observable absence of insolubles in the alcohol solvent) of the resulting rabeprazole sodium. The decolorizing agent can be any conventional decolorizing agent, including but not limited to, alumina, activated alumina, silica and charcoal. The decolorization temperature can be between about room temperature and below about the reflux temperature of the alcohol solvent. The decolorization temperature is advantageously room temperature (20-25 C).

Alternatively or additionally, the solution of the rabeprazole sodium acetone complex obtained in step i) can be filtered, if desired, in order to remove impurities (such as any insolubles in the alcohol solvent). The filtration temperature is preferably between room temperature and below the reflux temperature of the alcohol solvent, preferably room temperature. Thus, if there are impurities, e.g., observable impurities, the rabeprazole sodium acetone complex can be filtered, and then decolorized, or vice versa.

Rabeprazole sodium acetone complex can be obtained according to the known methods disclosed in examples 1 to 15 of U.S. Pat. No. 6,180,652. Other salts complexes can be made according to the methods described herein by obtaining salt complexes formed by using process conditions analogous to those used in forming the sodium acetone complex as described in U.S. Pat. No. 6,180,652.

It is well known that the purity levels of compounds can be enhanced by undertaking numerous iterations of purification steps. However, numerous iterations are not advantageous as they are not economically feasible, not only in terms of resources used in the purification steps but also in terms of lost yield which accompanies each purification step. The process of the invention is able to achieve high purity while maintaining high yield. Without wishing to be bound by any one particular theory, it is believed that this can be due at least in part to the fact that the process of the invention allows for the drying of the wet solid at temperatures lower than those previously used. Thus, drying of wet solids is advantageously employed in processes of the invention.

In one embodiment of the drying of the wet solid, the drying is accomplished at temperatures of less than 100° C. under reduced pressure. In another embodiment of the drying of the wet solid, the drying is accomplished at temperatures of about 40° C. to about 90° C. In yet another embodiment of the drying of the wet solid, the drying is accomplished at temperatures of about 60° C. to about 80° C.

In another embodiment of the invention, the yield of the process is at least about 75%. In a further embodiment, the yield of the process is at least about 85%.

A further embodiment of the invention includes amorphous rabeprazole sodium, and compositions thereof, having a particle size distribution wherein approximately 10% of the total volume is made of particles having a diameter less than approximately 2 μm, approximately 50% of the total volume is made of particles having a diameter less than approximately 12 μm and approximately 90% of the total volume is made of particles having a diameter less than approximately 39 μm. The invention further includes amorphous rabeprazole sodium having a surface area of approximately 2 to approximately 3 m²/g.

Yet another embodiment of the invention includes a large-scale (industrial scale) process to prepare rabeprazole sodium acetone complex from rabeprazole acid using aqueous NaOH in amounts less than the stoichiometric amounts used in example 33 of U.S. Pat. No. 5,045,552, preferably between approximately 0.75 and approximately 0.99 equivalents. The advantage of using less stoichiometric amounts of aqueous NaOH is the avoidance of an impurity at a retention time of 4.2 min. (HPLC) that appears when the reaction is carried out at large-scale in stoichiometric conditions.

The invention will now be further described by way of the following non-limiting examples. The following examples are given for the purpose of illustrating the present invention and shall not be construed as being limitations on the scope or spirit of the invention.

### EXAMPLES

### General Experimental Conditions

i. HPLC Method

The chromatographic separation was carried out in a Waters Symmetry C18, 5 μm, 4.6 mm×250 mm column.

The mobile phase was prepared by mixing 600 ml of methanol, with 400 ml of water and 10 ml of triethylamine. The pH was adjusted to 7.0 with phosphoric acid. The mobile phase was mixed and filtered through a 0.22 μm nylon membrane under vacuum.

The chromatograph was equipped with a 284 nm detector and the flow rate was 0.7 ml/min at 20-25° C. Tests samples (20 μl) were prepared by dissolving 25 mg of sample in 25 ml of methanol. The solution was freshly prepared.

ii. Particle Size Method

The particle size for amorphous rabeprazole sodium was measured using a Malvern Mastersizer S particle size analyzer with an MS1 Small Volume Recirculating unit attached. A 300RF mm lens and a beam length of 2.4 mm were used. Samples for analysis were prepared by dispersing a weighed amount of amorphous rabeprazole sodium (approximately 0.1 g) in 20 ml of Isopar G. The suspension was delivered drop-wise to a background corrected measuring cell previously filled with Isopar G until the obscuration reached the desired level. Volume distributions were obtained for three times. After completing the measurements, the sample cell was emptied and cleaned, refilled with suspending medium, and the sampling procedure repeated again. For characterization, the values of D₁₀, D₅₀ and D₉₀ (by volume) were specifically listed, each one being the mean of the six values available for each characterization parameter.

iii. Specific Surface Area Method

The BET (Brunauer, Emmett and Teller) specific surface area for amorphous rabeprazole sodium was measured using a Micromeritics ASAP2010 equipment. Samples for analysis were degassed at 100° C.-120° C. under vacuum for two hours. The determination of the adsorption of N₂ at 77° K was measured for relative pressures in the range of 0.07-0.2 for a weighed amount of amorphous rabeprazole sodium (i.e., approximately 0.5 g).

### Reference Example

### Preparation of 2-({[4-(3-methoxypropoxy)-3-methylpyridin-2-yl]methyl}sulfinyl)-1H-benzimidazole sodium salt acetone Complex (Rabeprazole sodium acetone Complex)

5.00 g (13.1 mmol) of Rabeprazole sodium obtained according to the known methods disclosed in example 33 of U.S. Pat. No. 5,045,552 was suspended in 25 ml of acetone. The suspension was stirred at 25° C. for 15 minutes. The solid was filtered and dried at 25° C. for 24 hours under reduced pressure to give 5.57 g of the title compound.

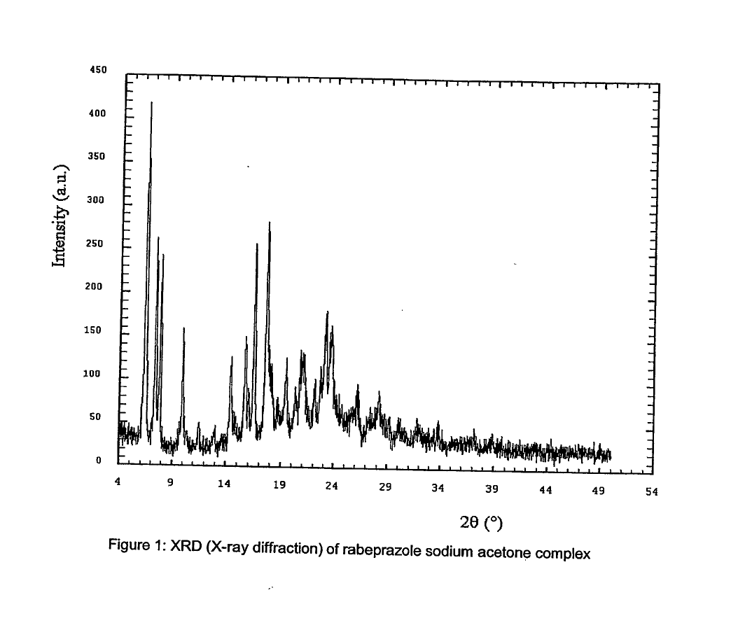

XRD (2Θ): The diffractogram is shown in FIG. 1.

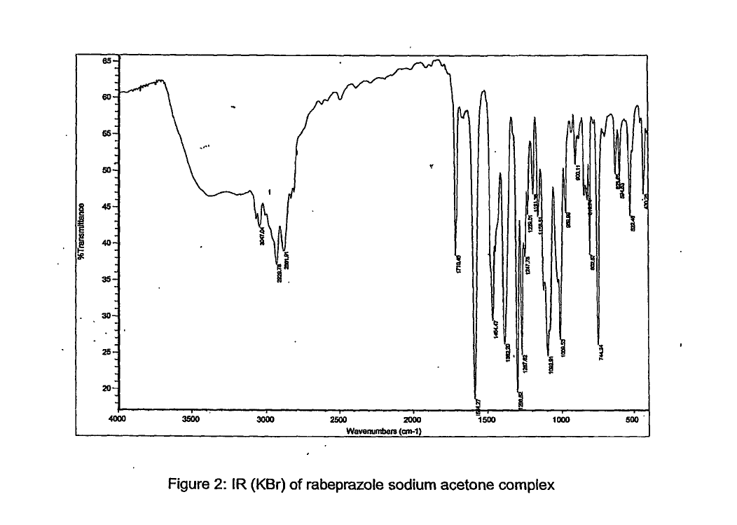

IR: The IR spectrum is shown in FIG. 2.

### Example 1

### Preparation of amorphous rabeprazole sodium by Suspending rabeprazole sodium acetone Complex in n-heptane

5.00 g (13.1 mmol) of rabeprazole sodium was suspended in 25 ml of acetone. The suspension was stirred at 25° C. for 30 minutes. The solid was filtered to obtain rabeprazole sodium acetone complex. This solid was suspended in 25 ml of n-heptane, stirred at 25° C. for 30 minutes and filtered. The solid obtained was suspended again in 25 ml of n-heptane, stirred at 25° C. for 30 minutes, filtered and dried at 25° C. for 24 hours under reduced pressure to give 4.35 g (11.4 mmol, yield 87%) of amorphous rabeprazole sodium.

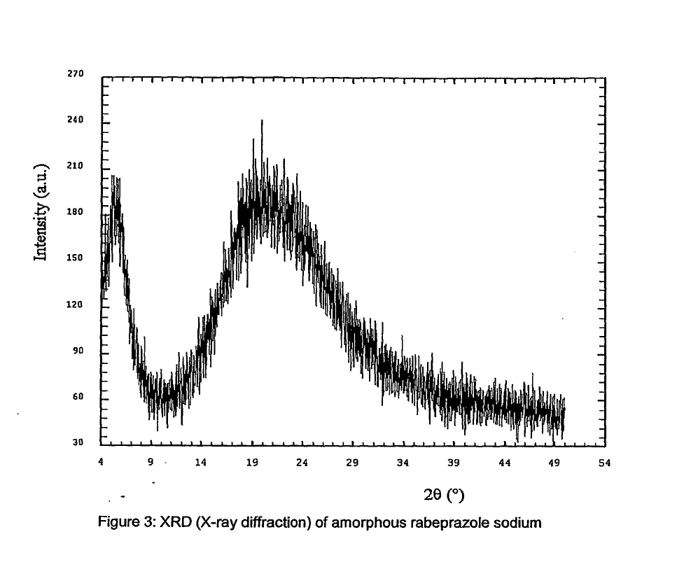

XRD (2Θ): The diffractogram is substantially identical to the diffractogram shown in FIG. 3.

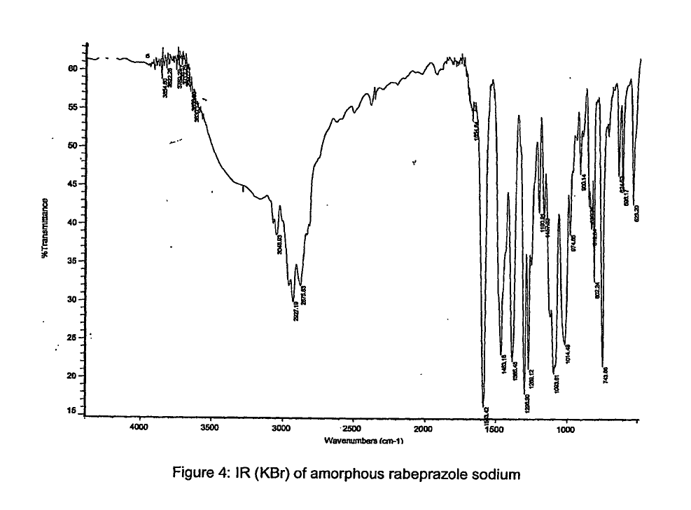

IR: The IR spectrum is substantially identical to the IR shown in FIG. 4.

HPLC (area %): 99.6%

### Example 2

### Preparation of amorphous rabeprazole sodium by Suspending rabeprazole sodium acetone Complex in tert-butyl methyl ether

5.00 g (13.1 mmol) of rabeprazole sodium was suspended in 25 ml of acetone. The suspension was stirred at 25° C. for 30 minutes. The solid was filtered to obtain rabeprazole sodium acetone complex. This solid was suspended in 25 ml of tert-butyl methyl ether, stirred at 25° C. for 30 minutes and filtered. The solid obtained was suspended again in 25 ml of tert-butyl methyl ether, stirred at 25° C. for 30 minutes, filtered and dried at 25° C. for 24 hours under reduced pressure to give 4.43 g (11.6 mmol, yield 89%) of amorphous rabeprazole sodium.

XRD (2Θ): The diffractogram is substantially identical to the diffractogram shown in FIG. 3.

IR: The IR spectrum is substantially identical to the IR shown in FIG. 4.

HPLC (area %): 99.4%

### Example 3

### Preparation of amorphous rabeprazole sodium by Suspending rabeprazole sodium acetone Complex Twice in n-heptane Followed by Distillation

10.00 g (26.2 mmol) of rabeprazole sodium was suspended in 50 ml of acetone. The suspension was stirred at 25° C. for 30 minutes. The solid was filtered to obtain rabeprazole sodium acetone complex. This wet solid was separated in three portions.

One portion of the wet solid was suspended in 40 ml of n-heptane and the solvent was distilled under vacuum to dryness. 40 ml of n-heptane was added and the solvent was partially distilled under vacuum. The solid was filtered and dried at at 25° C. for 24 hours under reduced pressure.

XRD (2Θ): The diffractogram is substantially identical to the diffractogram shown in FIG. 3.

IR: The IR spectrum is substantially identical to the IR shown in FIG. 4.

### Example 4

### Preparation of amorphous rabeprazole sodium by Dissolving rabeprazole sodium acetone Complex in methanol Followed by Distillation and Treatment with n-heptane and Distillation

25.00 g (65.5 mmol) of Rabeprazole sodium was suspended in 125 ml of acetone. The suspension was stirred at 25° C. for 30 minutes. The solid was filtered to obtain Rabeprazole sodium acetone complex. The solid was dissolved in 15 ml of methanol at 25° C. The solvent was distilled under vacuum to dryness. 100 ml of n-heptane was added and the solvent was partially distilled under vacuum until a solid precipitated. 100 ml of n-heptane was added and the solvent was partially distilled under vacuum. The suspension was stirred at 25° C. for 2 hours and a fraction of the solid was filtered. The following analysis of the wet solid were done:

XRD (2Θ): The diffractogram is substantially identical to the diffractogram shown in FIG. 3.

IR: The IR spectrum is substantially identical to the IR shown in FIG. 4.

The suspension was stirred at 25° C. for 17 hours. The solid was filtered and dried at 40° C. for 24 hours under reduced pressure to give 22.55 g (59.1 mmol, yield 90%) of the amorphous rabeprazole sodium.

XRD (2Θ): The diffractogram is substantially identical to the diffractogram shown in FIG. 3.

IR: The IR spectrum is substantially identical to the IR shown in FIG. 4.

HPLC (area %): 99.3%

### Example 5

### Preparation of amorphous rabeprazole sodium by Dissolving rabeprazole sodium acetone Complex in isopropanol Followed by Distillation and Treatment with n-heptane and Distillation

15.00 g (39.3 mmol) of rabeprazole sodium was suspended in 75 ml of acetone. The suspension was stirred at 25° C. for 30 minutes. The solid was filtered to obtain rabeprazole sodium acetone complex. To the solid 15 ml of isopropanol was added and the suspension was dissolved by heating at 40° C. The solvent was distilled under vacuum to dryness. Then, 60 ml of n-heptane was added and the solvent was distilled under vacuum to dryness. Then, 60 ml of n-heptane was added and the solvent was partially distilled under vacuum until a solid precipitated. The suspension was stirred at 25° C. for 20 hours. The solid was filtered and the following analysis of the wet solid were done:

IR: The IR spectrum is substantially identical to the IR shown in FIG. 4.

The solid was dried at 50° C. for 24 hours under reduced pressure to give 11.90 g (31.2 mmol, yield 79%) of the amorphous rabeprazole sodium.

XRD (2Θ): The diffractogram is substantially identical to the diffractogram shown in FIG. 3.

IR: The IR spectrum is substantially identical to the IR shown in FIG. 4.

HPLC (area %): 99.5%

### Example 6

### Preparation of amorphous rabeprazole sodium by Dissolving rabeprazole sodium acetone Complex in a Mixture of methanol and isopropanol Followed by Distillation and Treatment with n-heptane and Distillation

20.00 g (52.4 mmol) of rabeprazole sodium was suspended in 100 ml of acetone. The suspension was stirred at 25° C. for 30 minutes. The solid was filtered to obtain rabeprazole sodium acetone complex. The solid was dissolved in 9 ml of methanol at 25° C. and 20 ml of isopropanol was added. The solvent was distilled under vacuum to dryness. Then, 60 ml of n-heptane was added and the solvent was distilled under vacuum to dryness. Then, 60 ml of n-heptane was added and the solvent was distilled under vacuum to dryness. Finally, 30 ml of n-heptane was added. The suspension was stirred at 25° C. for 45 minutes and the solid was filtered. The following analysis of the wet solid were done:

IR: The IR spectrum is substantially identical to the IR shown in FIG. 4.

The solid was dried at 50° C. for 24 hours under reduced pressure to give 17.76 g (46.6 mmol, yield 89%) of the amorphous rabeprazole sodium.

XRD (2Θ): The diffractogram is shown in FIG. 3.

IR: The IR spectrum is shown in FIG. 4

HPLC (area %): 99.7%

### Example 7

When reproducing Example 6 at higher scale using a mixture ratio of methanol/isopropanol about 1:3 instead of about 1:2, the amorphous rabeprazole sodium was obtained typically having the following particle size distribution: D (v, 0.1): 1.7 to 1.8 μm; D (v, 0.5): 10.0 to 11.3 μm; D (v, 0.9): 34.1 to 38.9 μm; and typically having the following surface area: 2.1664±0.0918 to 2.9135±0.0138 m²/g

### Example 8

This example illustrates the large-scale preparation of rabeprazole sodium acetone complex from rabeprazole acid using aqueous NaOH in a relationship of 0.95 equivalents.

To a suspension of 38.81 Kg of rabeprazole acid (108 mol) in 143 L of water at 20-25° C., 5.43 L (102.8 mol) of sodium hydroxide solution 50% was added. The suspension was stirred for 1 hour and filtered. The filtrate was distilled under vacuum to dryness. 154 L of isopropanol was charged and the solvent was distilled under vacuum to dryness twice. 51 L of heptane was charged and the solvent was distilled under vacuum to dryness twice. The distillation residue was suspended in 103 L of acetone and the suspension was stirred at 20-25° C. for 8 hours. The solid was filtered yielding 42.80 Kg of rabeprazole sodium acetone complex. HPLC analysis did not show any impurity with a retention time of about 4.2 min. The product was purified by crystallization from acetone/THF mixtures.

Having thus described in detail various embodiments of the present invention, it is to be understood that the invention defined by the above paragraphs is not to be limited to particular details set forth in the above description as many apparent variations thereof are possible without departing from the spirit or scope of the present invention.

What is claimed is:

1. A process for the preparation of amorphous rabeprazole sodium comprising the steps of:
i) contacting rabeprazole sodium acetone complex with a first solvent system;
ii) filtering the solid from the solvent system used in step i) or distilling, totally or partially, the solvent system used in step i) under reduced or atmospheric pressure, to thereby obtain a residue;
iii) contacting the solid or the residue of step ii) with a second solvent system;
iv) filtering to obtain a wet solid from the solvent system used in step iii) or distilling, totally or partially, the solvent system used in step iii) under reduced or atmospheric pressure to thereby obtain a wet solid;
v) optionally repeating steps iii) and iv) one or more times; and
vi) drying the wet solid.

2. The process of claim 1, wherein the first solvent is a hydrocarbon solvent, an ether solvent, an alcohol solvent or mixtures thereof and the second solvent system is a hydrocarbon solvent or an ether solvent.

3. The process of claim 2, wherein the hydrocarbon solvent is n-hexane and n-heptane and isomers or mixtures thereof, cyclohexane, toluene or xylene; the ether solvents is tert-butyl methyl ether, cyclic ethers, tetrahydrofuran or 1,4-dioxane; and the alcohol solvent is C₁ to C₄ straight or branched chain alcohol solvents or mixtures thereof.

4. The process of claim 3, wherein the hydrocarbon solvent is n-heptane, the ether solvent is tert-butyl methyl ether and the alcohol solvent is methanol, 2-propanol or mixtures thereof.

5. A process for the preparation of amorphous rabeprazole sodium comprising the steps of:
i) suspending rabeprazole sodium acetone complex with a first solvent system;
ii) filtering the solvent system used in step i) or distilling, totally or partially, the solvent system used in step i) under reduced or atmospheric pressure to obtain a wet solid;
iii) optionally repeating steps i) and ii) one or more times; and
iv) drying the wet solid.

6. The process of claim 5 wherein the first solvent system is a hydrocarbon solvent, an ether solvent or mixtures thereof.

7. The process of claim 6, wherein the hydrocarbon solvent is n-hexane and n-heptane and isomers or mixtures thereof, cyclohexane, toluene or xylene and the ether solvent is tert-butyl methyl ether, cyclic ethers, tetrahydrofuran or 1,4-dioxane.

8. The process of claim 7, wherein the hydrocarbon solvent is n-heptane and the ether solvent is tert-butyl methyl ether.

9. A process for the preparation of amorphous rabeprazole sodium comprising the steps of:
i) dissolving rabeprazole sodium acetone complex with a first solvent system;
ii) optionally decolorizing and/or filtering the solution;
iii) distilling, totally or partially, the solvent system used in step i) under reduced or atmospheric pressure to obtain a residue;
iv) contacting the residue with a second solvent system;
v) distilling, totally or partially, the solvent system used in step iv) under reduced or atmospheric pressure;
vi) optionally repeating steps iv) and v) one or more times;
vii) filtering to obtain a wet solid; and
viii) drying the wet solid.

10. The process of claim 9, wherein the first solvent system is one or more alcohol solvent and the second solvent is a hydrocarbon solvent or an ether solvent.

11. The process of claim 10, wherein the one or more alcohol solvent is a C₁ to C₄ straight or branched chain alcohol solvents or mixtures thereof; the hydrocarbon solvent is a n-hexane and n-heptane and isomers or mixtures thereof, cyclohexane, toluene or xylene; and the ether solvent is a tert-butyl methyl ether, cyclic ethers, tetrahydrofuran, or 1,4-dioxane.

12. The process of claim 11, wherein the preparation of amorphous rabeprazole sodium comprises the steps of:
i) dissolving rabeprazole sodium acetone complex with a first solvent system comprising methanol and isopropanol to form a solution;
ii) decolorizing and filtering the solution;
iii) distilling totally, the solvent system used in step i) under reduced or atmospheric pressure to obtain a residue;
iv) contacting the residue with a second solvent system comprising heptane;
v) distilling, totally or partially, the solvent system used in step iv) under reduced or atmospheric pressure;
vi) repeating steps iv) and v) one or more times;
vii) filtering, thereby obtaining a wet solid; and
viii) drying the wet solid.

13. The process of claim 1, wherein the drying the wet solid is under reduced pressure at a temperature range selected from the group consisting of less than 100° C.; about 40° C. to about 90° C.; and about 60° C. to about 80° C.

14. The process of claim 5, wherein the drying the wet solid is under reduced pressure at a temperature range selected from the group consisting of less than 100° C.; about 40° C. to about 90° C.; and about 60° C. to about 80° C.

15. The process of claim 9, wherein the drying the wet solid is under reduced pressure at a temperature range selected from the group consisting of less than 100° C.; about 40° C. to about 90° C.; and about 60° C. to about 80° C.

16. The process of claim 1, wherein the drying temperature is about 60° C. to about 80° C. under reduced pressure and the yield is at least about 85%.

17. The process of claim 5, wherein the drying temperature is about 60° C. to about 80° C. under reduced pressure and the yield is at least about 85%.

18. The process of claim 9, wherein the drying temperature is about 60° C. to about 80° C. under reduced pressure and the yield is at least about 85%.

19. An amorphous rabeprazole sodium compound produced by any one of the processes of claims 1, 5 or 9.

20. The amorphous rabeprazole sodium compound of claim 19 with the X-ray diffraction pattern of FIG. 3.

21. The amorphous rabeprazole sodium compound of claim 19, wherein said amorphous rabeprazole sodium compound has a particle size distribution wherein approximately 10% of the total volume comprises particles having a diameter less than approximately 2 μm, approximately 50% of the total volume comprises particles having a diameter less than approximately 12 μm and approximately 90% of the total volume comprises particles having a diameter less than approximately 39 μm.

22. The amorphous rabeprazole sodium compound of claim 19, wherein said amorphous rabeprazole sodium compound has a surface area of approximately 2 to approximately 3 m²/g.
